(19) United States
(12) Patent Application Publication
(10) Pub. No.: US 2014/0371137 A1
(43) Pub. Date: Dec. 18, 2014

(54) PREPARATION OF PNA-6-AMINOGLUCOSAMINE CONJUGATES AS ANTIVIRAL AGENTS

(71) Applicant: Rutgers, The State University of New Jersey, New Brunswick, NJ (US)

(72) Inventors: Virendra Pandey, New Brunswick, NJ (US); Jean-Luc Decout, New Brunswick, NJ (US); Indrajit Das, New Brunswick, NJ (US); Jerome Desire, New Brunswick, NJ (US)

(21) Appl. No.: 14/304,560

(22) Filed: Jun. 13, 2014

Related U.S. Application Data
(60) Provisional application No. 61/834,648, filed on Jun. 13, 2013.

(51) Int. Cl. C12N 15/113; A61K 47/26

(52) U.S. Cl. CPC C12N 15/1132; A61K 47/26; C12N 2310/11; C12N 2310/3181; C12N 2310/351; C12N 2320/30
USPC 514/4.3; 530/322; 514/20.9; 514/3.7; 514/3.8; 514/19.3

(57) ABSTRACT

The present invention relates to methods and compositions pertaining to conjugates comprising a nucleic acid oligomer conjugated to a glucosamine or a derivative thereof which are useful for inhibiting the transcription of target nucleic acids. The conjugates of the invention exhibit advantageous bioavailability and readily penetrate cell membranes which make them useful for inhibiting translation of target mRNA in vivo.

### RELATED APPLICATIONS

This application claims priority to provisional application No. 61/834,648, filed on Jun. 13, 2013, the entire contents of which are incorporated herein by reference.

### FIELD OF THE INVENTION

The present invention pertains to the use of nucleic acid oligomer-glucosamine conjugates as therapeutic agents.

### BACKGROUND OF THE INVENTION

Nearly all disease states in multicellular organisms involve the action of proteins. Classic therapeutic approaches have focused on the interaction of proteins with other molecules in efforts to moderate the proteins' disease-causing or disease-potentiating activities. In newer therapeutic approaches, modulation of the production of proteins has been sought. A general object of some current therapeutic approaches is to interfere with or otherwise modulate gene expression. One method for inhibiting the expression of specific genes involves the use of oligonucleotides, particularly oligonucleotides that are complementary to a specific target messenger RNA (mRNA) sequence. Due to promising research results in recent years, oligonucleotides and oligonucleotide analogs are now accepted as therapeutic agents holding great promise for therapeutic and diagnostic methods. Oligonucleotides and their analogs can be designed to have particular properties. A number of chemical modifications have been introduced into oligomeric compounds to increase their usefulness as therapeutic agents. Such modifications include those designed to increase binding affinity to a target strand, to increase cell penetration, to stabilize against nucleases and other enzymes that degrade or interfere with the structure or activity of the oligonucleotide, to provide a mode of disruption (terminating event) once the oligonucleotide is bound to a target, and to improve the pharmacokinetic properties of the oligonucleotide.

Peptide nucleic acids (PNAs) are a class of antisense DNA analogues first synthesized by Nielsen and colleagues in 1991. The PNA molecules, devoid of sugar phosphate backbone and charges under physiological conditions, have been shown to display high affinity for complementary sequences on RNA and DNA both in single and double stranded forms. PNA are highly stable and remained uncleaved when incubated with blood or cell lysate from human and bacterial cells. Initial expectations held that PNAs would quickly enter the field of antisense as genespecific, nontoxic, and nonimmunogenic agents. However, problems associated with solubility and poor cellular uptake of this class of compounds hampered developments in this direction. The synthesis of modified PNAs or PNA conjugates presents new means of improving their solubility and cellular uptake.

During the past several years, the applicants' research has mainly focused on designing sequence specific inhibitors for blocking viral replication. Peptide nucleic acids (PNAs) are DNA analogs that have received a lot of attention in the community due to their high affinity for complementary sequences on RNA and DNA both in single and double stranded forms. However, their therapeutic potential for gene-specific, nontoxic, and non-immunogenic therapy has been limited as nucleic acid binding agents due to poor uptake into mammalian cells. The synthesis of modified PNA or PNA conjugates presents new means of improving the cellular uptake and improving their functional efficacy. We have demonstrated that cell penetrating peptides conjugated to PNAs targeting the transactivation response element (TAR), primer binding site (PBS) and A-loop region of HIV-1 RNA are potent inhibitor of HIV-1 replication and viral production when supplemented in HIV-1 infected cell culture.

Despite these advances, a need exists for nucleic acid oligomers having improved bioavailability.

### SUMMARY OF THE INVENTION

In an aspect of the present invention, there is provided a compound comprising a nucleic acid oligomer and a glucosamine conjugated thereto.

In another aspect, the present invention provides a method of introducing a nucleic acid into a cell comprising contacting said cell with a conjugate compound of the invention.

In another aspect, the present invention provides a method of inhibiting the transcription or translation of a target nucleic acid in a cell comprising introducing into said cell a conjugate compound of the invention wherein the nucleic acid of the conjugate compound hybridizes to the target nucleic acid.

In another aspect, the present invention provides a method of treating a disease or disorder in a mammal mediated by a protein or nucleic acid comprising administering to said mammal an effective amount of a conjugate compound of the invention wherein the nucleic acid of the conjugate compound hybridizes to the nucleic acid mediating said disease or disorder or to the nucleic acid encoding the protein mediating said disease or disorder.

In another aspect, the present invention provides a method of detecting a nucleic acid in a cell comprising contacting said cell with a conjugate compound of the invention wherein said conjugate compound comprises a detectable label.

### BRIEF DESCRIPTION OF THE DRAWINGS

FIG. 1 shows the structure of the anti-TAR PNAs 1a and 1b, their glucosamine and neamine conjugates 2a, 2b and 3;

FIG. 2 depicts the binding affinity of PNA_{TAR}-glucosamine conjugate to its target sequence;

FIG. 3 illustrates results of flow cytometry analysis of uptake of anti-HIV-1 PNA_{TAR}-glucosamine conjugate;

FIG. 4 is a representation of a primer extension assay with TAR-RNA template in the presence PNA_{TAR}-glucosamine conjugate; and

FIG. 5 is a graphical depiction of the dose effect curve of virucidal activity of anti-HIV-1 PNA_{TAR}-glucosamine conjugate.

FIG. 6. Organization of HCV Genome.

FIG. 7. Structure of HCV 5′-NTR.

FIG. 8. Structural Domains of HCV 3′-NTR.

FIG. 9. Structure of PNA and PNA-neamine Conjugate.

FIG. 10. Stoichiometry of binding of PNA-glucosamine conjugate to its target sequence: (A) The PCR product corresponding to domains III and IV of 5′NTR and the 36 nucleotide of the N-terminal coding sequence of the HCV core were transcribed to generate 244-base runoff transcripts using a T7 transcription kit from Roche Applied Sciences. The 20 nM of internally 32P labeled HCV 5′NTR RNA transcript was incubated either with various concentrations of PNA-glucosamine conjugate complementary to sequence 352-338 in domain IV of the 5′NTR or (B) scramble PNA-glucosamine conjugate. Binding of the PNA-glucosamine conjugate (PNAHCV 352-338) to its target sequence was evaluated by gel electrophoretic mobility shift analysis (EMSA). Lane 1, control without PNA conjugate; lane 2, 10 nM of PNA conjugate; lane 3, 15 nM of PNA conjugate; lane 4, 20 nM of conjugate, lane 5, 25 nM of conjugate.

FIG. 11. Uptake of PNA-glucosamine conjugate by Huh7.5 cells: Huh7.5 cells grown in Dulbecco's modified medium with 10% fetal calf serum (FCS) were washed with PBS and resuspended in the same medium (4×10⁶ cells/ml) containing 2% FCS. Cells (0.5 ml) were liquated in 12-well microtiter plates at 2×10⁶ cells per well and incubated at room temperature with varying concentrations (50-500 nM) of fluorescein-tagged PNA-glucosamine conjugate. After 6 h of incubation, the fluorescence signal /10,000 cells was obtained using fluorescent flow cytometry (FACscan).

FIG. 12 Affinity capture of HCV (+) RNA-protein complex from MH-14 cells. The biotinylated PNA-glu-HCV-Core conjugate complementary to nucleotide sequence 342-356 of HCV (+) strand RNA was incubated with (A) MH14 cells (HCV positive) or (B) cured MH14 cells (HCV negative) as described in the Methods. The conjugate penetrated the cells and bound to its target sequence was captured from cell lysate on paramagnetic streptavidin beads. The beads were washed and suspended in SDS gel loading buffer, heated at 90° C. for 5 min prior to magnetic separation of beads from eluted proteins and supernatant was resolved by SDS-PAGE and visualized by staining the gel with Sypro ruby. Lane 1, cell lysate; lane 2, affinity captured HCV RNA-protein complex bound to biotinylated anti-HCV PNA-glucosamine conjugate immobilized on streptavidin beads; Lane 3, cell lysate supernatant flow thru of the streptavidin beads; lanes 4-6, bead washes with 0.5 M NaCl in reticulocyte buffer.

FIG. 13. Inhibition of viral translation and replication by anti-HCV PNA-glucosamine conjugate. MH14 cells carrying constitutionally replicating HCV subgenomic replicons were grown in medium supplemented with different concentrations of the conjugate targeted to sequence 352-338 in domain IV of HCV 5′NTR. Cells were harvested after 72 h and analyzed for viral protein by Western blot (A), then for viral RNA by RT PCR (B). Lane 1, control without the conjugate; lanes 2-4 contain, respectively, 50 nM, 100 nM and 200 nM of anti-HCV PNA-glucosamine conjugate.

FIG. 14. Agarose gel electrophoresis of reaction products catalyzed in cell free extract. The subconfluent MH14 cells were incubated for 8 hours in the absence and presence of 250 nM of PNA-glucosamine-RNA1-16 conjugate targeted to 3′ terminal region of HCV (−) strand RNA. We lysed the cells and prepared the replication lysate for endogenous replication activity with [α-32P] CTP (30 μCi; 800 Ci/mmol) essentially as described by Ali et al, (2002). The RNA products of the replication reaction were analyzed on both (A) native and (B) denaturing agarose gel and visualized by a Phosphorlmager. Lane 1 shows no product in cured MH14; lanes 2 and 3 show the RNA products of HCV replication activity in the cell-free extract of MH14 cells pre-incubated in the absence and absence of PNA-glucoHCV1-16 conjugate, respectively; Lane 4 represents in-vitro transcribed 7 kb HCV replicon as the molecular size marker.

### DETAILED DESCRIPTION OF INVENTION AND PREFERRED EMBODIMENTS

The present invention is directed to a compound comprising a nucleic acid oligomer and a glucosamine or neamine conjugated thereto. The compounds of the invention are alternatively referred to as “conjugate compounds” herein. An advantage of the present invention is increased biodelivery of the nucleic acid oligo into cells. A glucosamine includes without limitation 6-aminoglucosamine. In a particular embodiment, the glucosamine is 6-aminoglucosamine.

In a particular embodiment, the nucleic acid oligomer (aka “oligo”) of the conjugates is RNA, DNA or analogs thereof which are capable of hybridizing in a sequence specific manner to a target nucleic acid. DNA and RNA analogs may comprise one or more sugar, base or backbone modifications at one or more nucleoside.

In a particular embodiment a PNA-Glucosamine conjugate is provided which is useful for blocking translation of nucleic acids encoding proteins, in particular, viral RNA. This nucleic acid portion of the compounds may be sequence-specific designed to selectively target the conserved regulatory elements of any of the infectious RNA viruses in order to block their translation and replication. Another advantage of the present invention is increased biodelivery and antiviral efficacy of PNA due to its conjugation with a glucosamine (neosamine).

In an embodiment, the conjugate nucleic acid is from about 10 to 50 nucleosides in length. In another embodiment, the conjugate nucleic acid is from about 10 to 30 nucleosides in length. In another embodiment, the conjugate nucleic acid is from about 10 to 25 nucleosides in length. In another embodiment, the conjugate nucleic acid is from about 15 to 20 nucleosides in length. In another embodiment, the conjugate nucleic acid is about 15 in length. In another embodiment, the conjugate nucleic acid is about 20 nucleosides in length.

In an embodiment, the conjugate nucleic acid optionally comprises one or more nucleosides wherein the sugar group has been modified. Such sugar modified nucleosides may impart enhanced nuclease stability, increased binding affinity or some other beneficial biological property to the antisense compounds. In certain embodiments, nucleosides comprise a chemically modified ribofuranose ring moieties. Examples of chemically modified ribofuranose rings include without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R1)(R)2 (R═H, C1-C12 alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group). Other examples of nucleosides having modified sugars include without limitation nucleosides comprising 5′-vinyl, 5′-methyl (R or S), 4′-S, 2′-F, 2′-OCH3 and 2′-O(CH2)2-O—CH3 substituent groups. The substituent at the 2′ position can also be selected from allyl, amino, azido, thio, O-allyl, O—C1-C10 alkyl, OCF3, O(CH2)2SCH3, O(CH2)2-O—N(Rm)(Rn), and O—CH2-C(O)—N(Rm)(Rn), where each Rm and Rn is independently H or substituted or unsubstituted C1-C10 alkyl. In certain embodiments, the ribosyl ring is replaced with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring. Many other bicyclo and tricyclo sugar surrogate ring systems are also know in the art that can be used to modify nucleosides for incorporation into antisense compounds (see for example review article: Leumann, J. C, Bioorganic & Medicinal Chemistry, 2002, 10, 841-854).

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-Me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278). Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases such as 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Heterocyclic base moieties may also include those in which the purine or pyrimidine base is replaced with other heterocycles, for example 7-deazaadenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2-aminopropyladenine, 5-propynyluracil and 5-propynylcytosine.

In an embodiment, the conjugate nucleic acid oligo comprises one or more backbone modifications. For example, the backbone may be a phosphorothioate backbone or other heteroatom containing backbone such as —CH2-NH—O—CH2-, —CH2-N(CH3)-O—CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester backbone is represented as —O—P—O—CH2-) of the above referenced U.S. Pat. No. 5,489,677, and the amide backbones of the above referenced U.S. Pat. No. 5,602,240. Also suitable are oligonucleotides having morpholino backbone structures of the above-referenced U.S. Pat. No. 5,034,506.

In an embodiment, the conjugate nucleic acid oligo contains both the sugar and internucleoside linkage (i.e. the backbone) modifications. In a particular embodiment, one or more nucloesides of conjugate nucleic acid oligos is a peptide nucleic acid (PNA) in which the sugar-backbone is replaced with an amide containing backbone, in particular an aminoethylglycine backbone. The nucleobases are retained and are bound directly or indirectly to aza nitrogen atoms of the amide portion of the backbone. PNA compounds are described in U.S. Pat. Nos. 5,539,082; 5,714,331; and 5,719,262. In a particular embodiment, the conjugate nucleic acid is a PNA having the sequence TCCCAGGCTCAGATCT.

In an embodiment the conjugate nucleic acid oligo comprises one or more bicyclic nucleic acid (BNA). Examples of BNAs include without limitation nucleosides comprising a bridge between the 4′ and the 2′ ribosyl ring atoms. In certain embodiments, BNA nucleosides comprises one of the formulas: 4′-(CH2)-O-2′ (LNA); 4′-(CH2)-S-2; 4′-(CH2)-O-2′ (LNA); 4′-(CH2)2-O-2′ (ENA); 4′-C(CH3)2-O-2′ (see PCT/US2008/068922); 4′-CH(CH3)-O-2′ and 4′-CH(CH2OCH3)-O-2′ (see U.S. Pat. No. 7,399,845, issued on Jul. 15, 2008); 4′-CH2-N(OCH3)-2′ (see PCT/US2008/064591); 4′-CH2-O—N(CH3)-2′ (see published U.S. Patent Application US2004-0171570, published Sep. 2, 2004); 4′-CH2-N(R)—O-2′ (see U.S. Pat. No. 7,427,672, issued on Sep. 23, 2008); 4′-CH2-C(CH3)-2′ and 4′-CH2-C(═CH2)-2′ (see PCT/US2008/066154); and wherein R is, independently, H, C1-C12 alkyl, or a protecting group. Each of the foregoing BNAs include various stereochemical sugar configurations including for example alpha-L-ribofuranose and beta-D-ribofuranose (see PCT international application PCT/DK98/00393, published on Mar. 25, 1999 as WO 99/14226).

In a particular embodiment, the conjugate compound of the present invention is selected from the group consisting of:

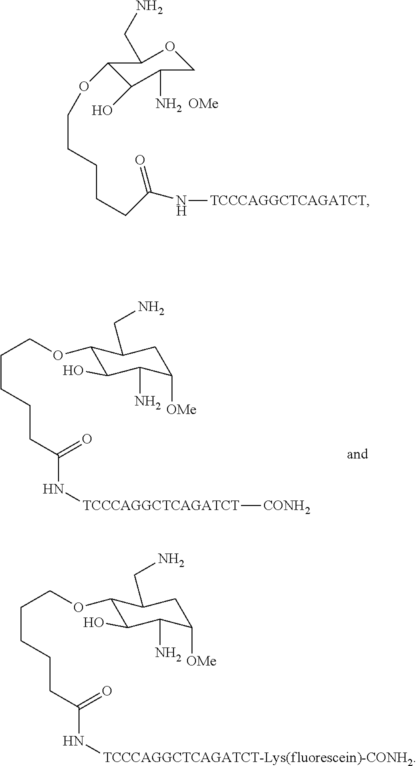

In another aspect there is provided a conjugate compound which is

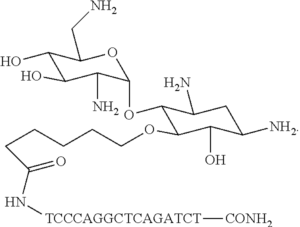

In another aspect, the present invention provides a method of introducing a nucleic acid into a cell comprising contacting said cell with a conjugate compound of the invention.

In another aspect, the present invention provides a method of inhibiting the transcription or translation of a target nucleic acid in a cell comprising introducing into said cell a conjugate compound of the invention wherein the nucleic acid of the conjugate compound hybridizes to the target nucleic acid.

In another aspect, the present invention provides a method of treating a disease or disorder in a mammal mediated by a protein or nucleic acid comprising administering to said mammal an effective amount of a conjugate compound of the invention wherein the nucleic acid of the conjugate compound hybridizes to the nucleic mediating said disease or disorder or to the nucleic acid encoding the protein mediating said disease or disorder. In a particular embodiment, the disease or disorder is a viral infection in a mammal. In a particular embodiment, the viral infection results from infectious RNA viruses, specifically those that replicate and complete their life cycle in the cytosol. In an embodiment, the virus is HCV. A PNA-Glucosamine conjugate may be used to block the translation and replication of infectious RNA viruses. This conjugate compound can be designed to selectively target the conserved regulatory elements of any of the infectious RNA viruses in order to block their translation and replication. An advantage of the present invention is increased biodelivery and antiviral efficacy of PNA due to its conjugation with 6-aminoglucosamine. In one embodiment, the present invention provides a method of preventing or treating infections which result from infectious RNA viruses, specifically those that replicate and complete their life cycle in the cytosol. Sequence specific PNA conjugated with 6-aminoglucosamine is efficiently taken up by cells. The conjugate internalized in the cell remains localized in the cytosol. This property of the conjugate makes it an excellent candidate to target critical regions of most of the infectious RNA viruses that replicate and complete their life cycle in the cytosol. In another embodiment, the sequence specific PNA-glucosamine conjugate targets either positive sense and negative sense RNA viruses in the cells. In a further embodiment, the conjugate compounds of invention target double stranded RNA viruses. The conjugates of the invention are effective for entering cells because the oligos are chargeless molecule; therefore, it is able to penetrate and bind to the target sequence in the duplex region of RNA and DNA.

In another embodiment, the conjugate can be used to irreversibly block function of a targeted region of RNA and block virus replication and translation. The binding of the conjugate to its target sequence in the cell is nearly irreversible under physiological conditions. Thus the function of targeted region of the RNA viruses could be invalidated resulting in blockage of virus replication and translation. In yet another embodiment of the present invention, the sequence specific conjugate can be used to target mRNAs to block their translation in the cytosol.

In another embodiment of the present invention, the conjugate is designed to target the function of RNA template component of the telomerase, an enzyme that is responsible for survival of many of the cancer cells. Accordingly, the conjugate compounds may be used in method for treating cancer. The telomere consists of a number of TTAGGG repeat sequences that shorten after every cell division. In most somatic tissues, telomerase is expressed at very low levels or not at all, as cells divide, telomeres shorten. Short telomeres may be a signal for cells to senesce (stop dividing). Cancer cells express high level of telomerase that helps them to survive by extending the telomere ends. The telomerase enzyme extends the telomeric end of the chromosome by copying its own RNA template (AAUCCC) complementary to the telomeric sequences. The PNA glucosamine conjugate can irreversibly bind to this RNA template and block the function of enzyme leading to senesce and death of cancer cells.

In another aspect of the invention, there is provided treatments of 100 mg PNA-CPP conjugate/kg body weight. The conjugate is highly stable and completely resistance to cellular enzymes. We have earlier shown that PNA conjugated with cell penetrating peptide (CPP) are non-toxic in mouse model (repeat doses of 100 mg PNA-CPP conjugate/kg body wt was well tolerated without any toxic effect. It is therefore expected that PNA-glucosamine conjugate will display similar non-toxic profile in mouse model.

In another embodiment, the present invention comprises a method for modulating the activity of a nucleic acid molecule comprising contacting one or more nucleic acid molecules with a peptide nucleic acid-glucosamine conjugate which hybridizes with at least one nucleic acid molecule of the one or more nucleic acid molecules so that the function of the at least one nucleic acid molecule is modulated.

In yet another embodiment, the present invention provides a method for preventing or treating a disease caused by RNA viruses comprising administering to a patient with a disease associated with RNA viruses an effective amount of a peptide nucleic acid-glucosamine conjugate which hybridizes with the targeted critical regions of viral RNA genome so that the function of the viral genome is modulated and the disease associated with said virus is prevented or treated.

The sequence-specific PNA-neamine conjugate is a highly stable molecule that rapidly penetrates cells and remains localized at the site of HIV-1 translation and replication in the cytosol. Therefore, this conjugate can be designed to selectively target the conserved regulatory elements of any of the infectious RNA viruses and block translation and replication. As part of the present invention and in continuation of our work, we have further improved the biodelivery and antiviral efficacy of PNA by conjugating with aminoglucosamine. The sequence of PNA could be designed to target any critical regions of RNA viruses including HIV-1 and hepatitis C virus. In the present study, we targeted transactivating response (TAR) element of HIV-1 RNA to block Tat mediated transactivation of HIV-1 transcription. The sequence of PNA in this study was.

6-Aminoglucosamine is a small aminoglycoside constituted of only one sugar ring. The 6-aminoglucosamine core was conjugated to peptide nucleic acids (PNA) targeting the HIV TAR RNA through introduction of convenient protecting groups for the amino and hydroxyl functions. The synthesis and study of the corresponding fluorescent PNA conjugate showed that the presence of the 6-aminoglucosamine core in the PNA conjugates permits a very efficient cellular uptake. The PNA-glucosamine conjugate was able to penetrate in the cells and remained localized in cytosol. It strongly inhibited HIV-1 replication when supplemented in HIV-1 infected cell culture. The conjugate represents an excellent candidate for targeting infectious RNA viruses, especially those that replicate in the cytosol.

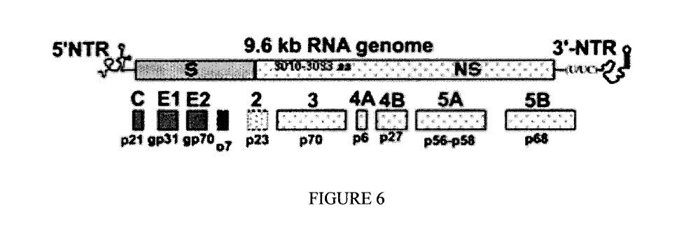

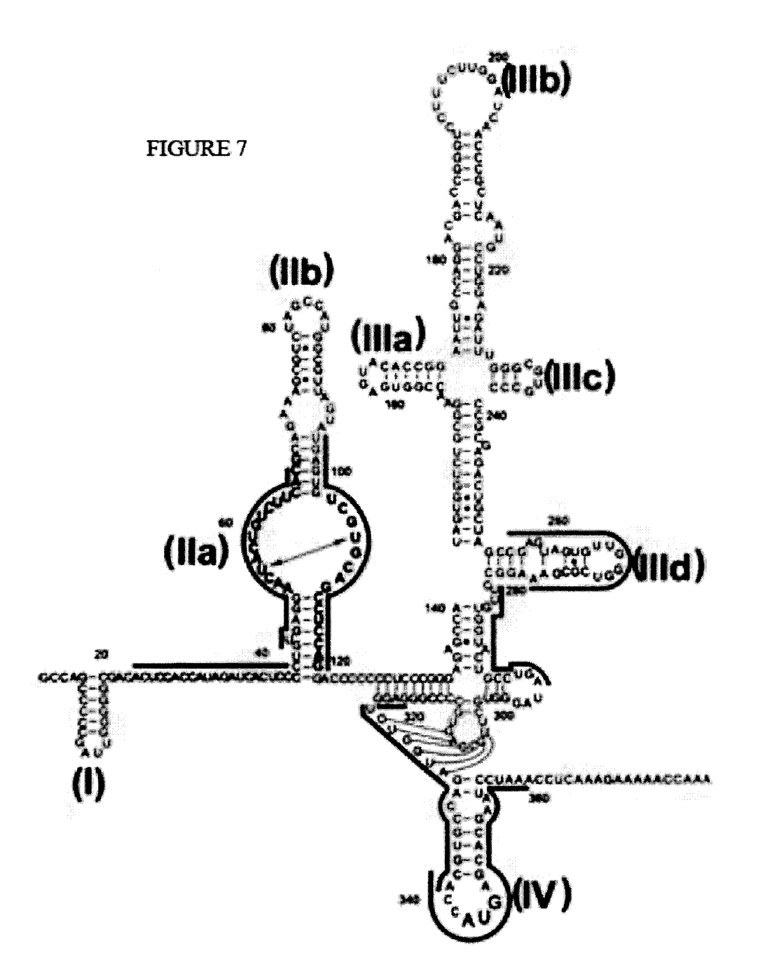

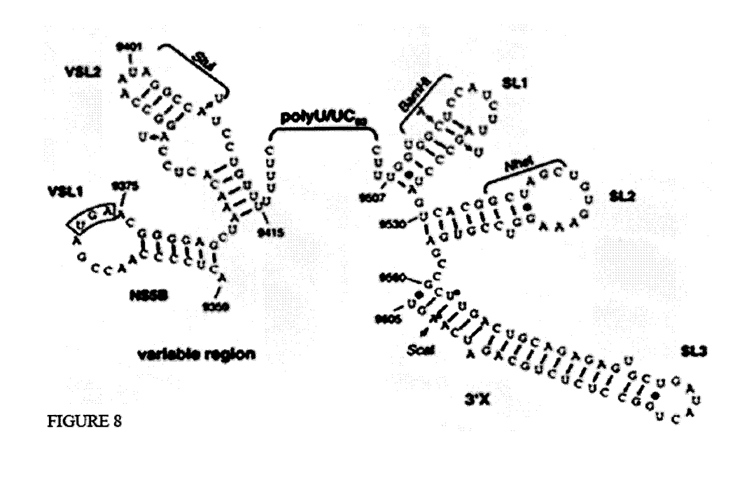

The 5′ and 3′ NTR of the HCV genome: The HCV RNA genome, which is approximately 9.6 kb long, contains a single long, open reading frame that encodes a polyprotein of approximately 3,000 amino acids. The structural proteins (C, E1, and E2) are located at the N-terminal portion, followed by non-structural proteins (NS2, NS3, NS4A, NS4B, NS5A, and NS5B) (FIG. 6). The first 341 nucleotides of HCV genomic RNA comprise the NTR and contain multiple AUG codons upstream of the translational start site. A second 247-nucleotide-long NTR is present at the 3′ terminus (3′ NTR) of the viral genome, which is essential for viral replication. The 5′NTR and 2′NTR are highly structured RNA elements containing multiple conserved regulatory sequences that are essential for translation and replication. The 5′ NTR folds into four distinct stem-loop domains (FIG. 7; Honda et al., 1999). Domains II and IV comprise an internal ribosome entry site (IRES) that is responsible for cap-independent initiation of the translation of the viral polyprotein. Domains I, II, and III contain overlapping replication signals that are required for optimal replication of the viral RNA in cell culture. These important conserved sites on the viral genome could be targeted by sequence specific PNA-glucosamine conjugate for blocking both HCV translation and replication. The 3′NTR also contains three structurally distinct domains: a very highly conserved 98-nucleotide 3′-terminal segment that forms three stem-loop structures, SL1, SL2 and SL3 (FIG. 8) in the 3′-to-5′ direction; a lengthy poly U/UC tract; and an upstream variable region (VR). Both the poly U/UC tract and the 98-nt conserved segment are required for infectivity and viral replication. These regions could be targeted with a new class of sequence-specific PNA-glucosamine conjugate for blocking HCV replication.

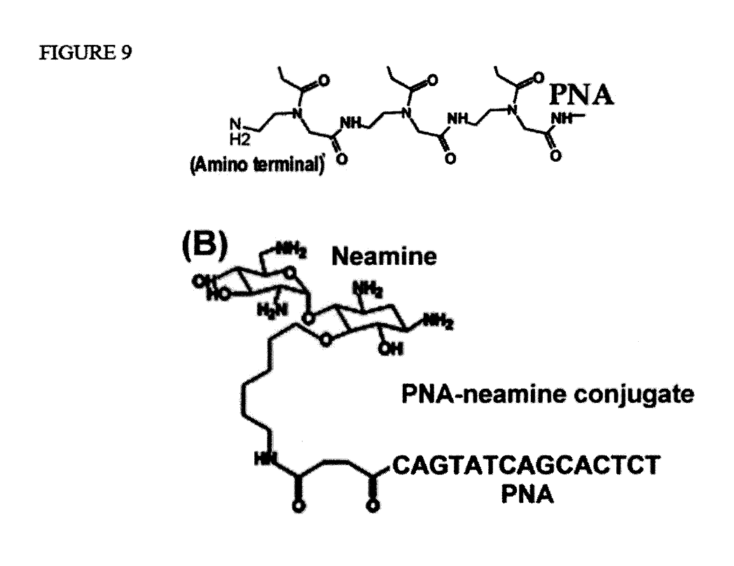

PNAs as potential antiviral agents: A class of novel DNA mimic, peptide nucleic acid (PNA), was first synthesized in 1991 (Nielsen et al., 1991). PNAs have no sugar-phosphate backbone, in which bases are linked via peptide bonds (FIG. 9A). We previously demonstrated that sequence-specific PNA is an excellent candidate for targeting critical regions of HIV-1 RNA to block their functions in cell culture systems. The cellular uptake and antiviral efficacy of sequence-specific PNA is significantly enhanced upon conjugation with cell-penetrating peptide. We found that anti-HIV-1 PNA-peptide conjugates have strong antiviral and virucidal activities, and favorable pharmacokinetic behavior, and are nearly non-toxic, even at concentrations as high as 300 mg/kg of body weight. However, since HIV-1 replicates via DNA intermediate, which is integrated in the host genome, the anti HIV-1 PNA, like other anti-HIV drugs could not cure the cell of HIV-1 infection. PNA conjugated with cell penetrating peptide such as penetratin or Tat peptide are mainly localized in the nucleus and therefore cannot be used to target RNA viruses such as HCV which replicates in the cytosol. Accordingly, we have designed a novel approach in which PNA is conjugated with the glucosamine. This conjugate which is primarily localized in the cytosol. Therefore, HCV RNA that replicates in the cytosol should be highly susceptible to anti-HCV PNA-glucosamine conjugates which effectively penetrate the cells and bind to their target sequence on HCV RNA.

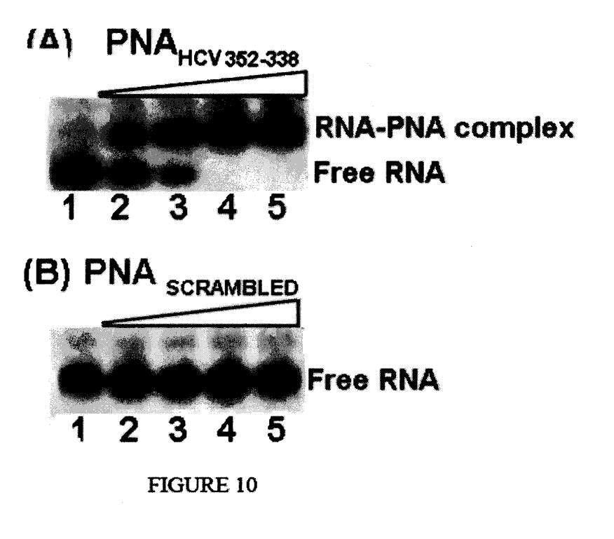

Target specificity of antisense PNA: Using a PNA-glucosamine conjugate targeted to the translation initiation window (352-338: TTCGTGCTCATGGTG) in Domain IV of HCV 5′NTR, we have shown that it has great specificity for the targeted sequence. The PNA-glucosamine conjugate (PNAHCV353-338) forms a tight complex with HCV RNA at a stoichiometric ratio (FIG. 10). At 1:0.5 and 1:0.75 ratios of target RNA to PNA conjugate, the respective gel retardation of the labeled RNA was 50 and 75% (FIG. 10A, lanes 2 and 3), while a complete gel shift of the target RNA was noted at stoichiometric ratios of 1:1 or greater (FIG. 10A, lanes 4 and 5).

In another aspect of the invention, there is provided a pharmaceutical composition comprising a conjugate compounds of the invention and pharmaceutically acceptable diluent or carrier. The conjugate compounds may be admixed, encapsulated, further conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and U.S. Pat. No. 5,595,756.

Administration of conjugate compounds of the invention may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients. Emulsions are typically heterogeneous systems of one liquid dispersed in another in the form of droplets usually exceeding 0.1μ in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860. The pharmaceutical formulations and compositions of the present invention may also include surfactants. The use of surfactants in drug products, formulations and in emulsions is well known in the art. Surfactants and their uses are further described in U.S. Pat. No. 6,287,860.

The formulation of therapeutic compositions and their subsequent administration (dosing) is within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

In another aspect, the present invention provides a method of detecting a nucleic acid in a cell comprising contacting said cell with a conjugate compound of the invention wherein said conjugate compound comprises a detectable label. In such methods, unbound labelled conjugate compound is removed and the label on the target bound conjugate is detected thereby indicating the presence of the target nucleic acid. In a particular embodiment, the detectable label is fluorescein, rhodamine, coumarin, dyes, and radioisotopes. In a particular embodiment, the label is fluorescein.

In a further aspect, the present invention provides a method for producing peptide nucleic acid-glucosamine conjugates comprising the steps of: 1) synthesis of a glucosamine derivative carrying attached to an oxygen atom a linking arm with a terminal carboxylate group allowing the conjugation to the PNA and 2) synthesis of the conjugate with a) protection of amino functions of the glucosamine moiety with an acid-labile protecting group; b) coupling of the PNA to the carboxylate group of the glucosamine derivative; c) deprotection of the amino functions and cleavage of the peptide nucleic acid-glucosamine conjugate from a solid support with an acid.

### EXAMPLES

The present invention is described more fully by way of the following non-limiting experimental examples. Modifications of these examples will be apparent to those skilled in the art. First, the 6-aminoglucosamine derivative A was synthesized from widely available and cost effective N-acetylglucosamine according to the following steps.

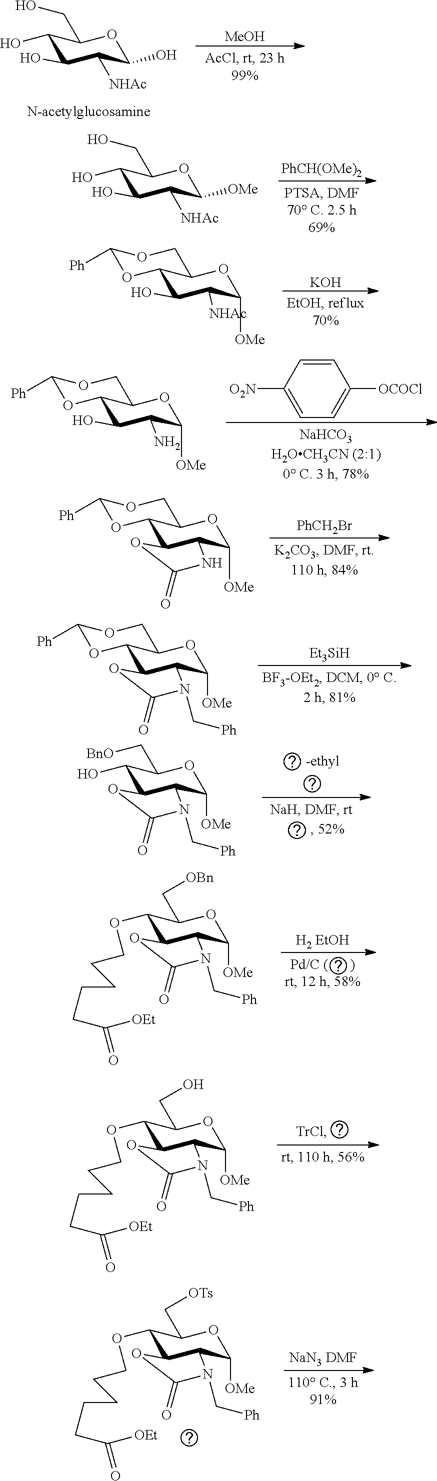

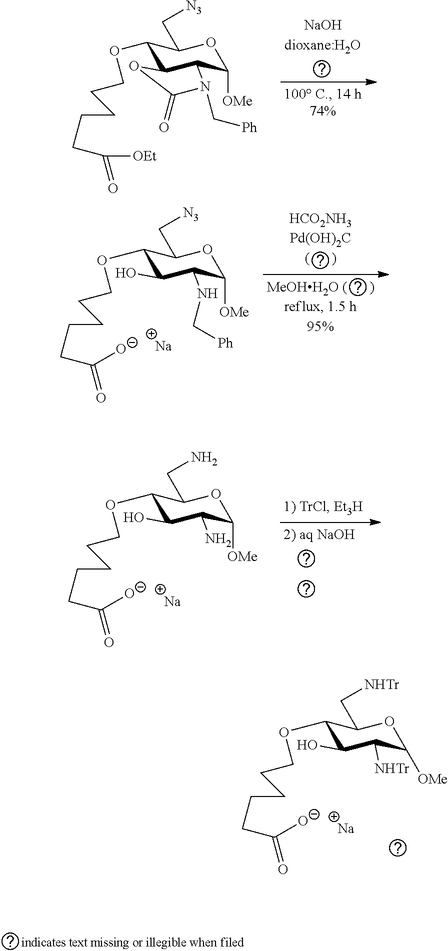

The sodium salt A was converted to the triethylammonium salt B which was coupled to the PNA attached to its solid support of synthesis. The conjugate was obtained after deprotection and cleavage from the solid support in TFA (purification by HPLC).

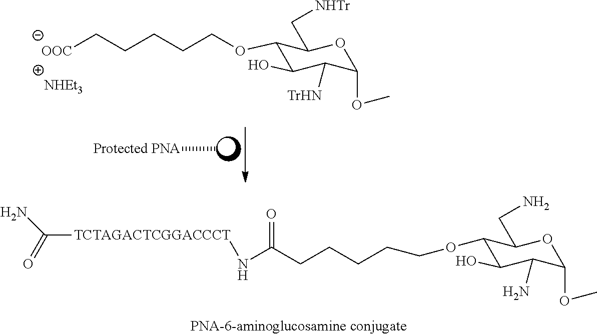

### Synthetic Scheme of PNA-Glucosamine Conjugate

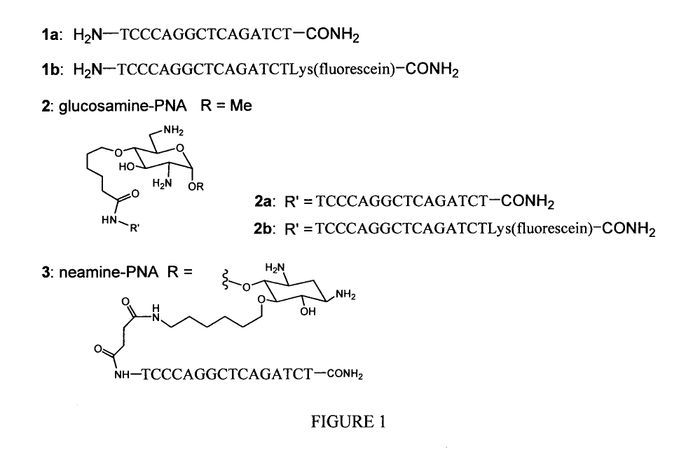

FIG. 1 depicts the structure of the anti-TAR PNAs 1a and 1b, their glucosamine and neamine conjugates 2a, 2b and 3.

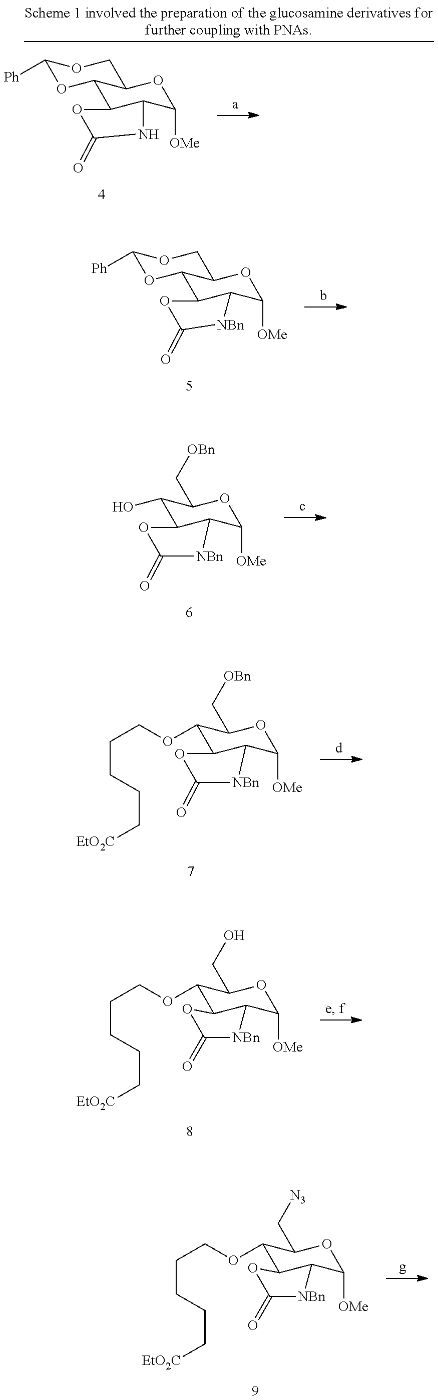

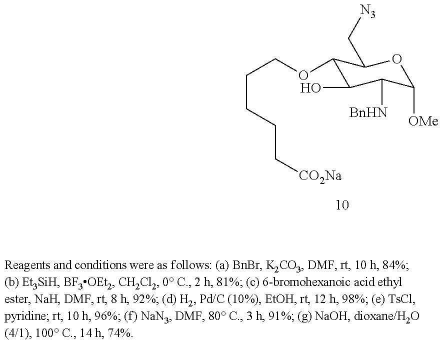

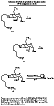

Glucosamine Derivatives for Coupling with PNA

For general applications, all reagents were used as purchased from suppliers without further purification. The protected 16-mer PNA oligomers were purchased from Eurogentec. DMF was distilled in the presence of CaH₂, and stored under argon atmosphere prior to use. Thin layer chromatographies were performed on silica gel (Alugram Sil G/UV₂₅₄) or Alumina gel (Alugram Alox N/UV₂₅₄) from Macherey-Nagel and spots were detected either by UV absorption or by charring with ninhydrin. HPLC purifications were carried out on a C18 reversed-phase column (Macherey-Nagel, 10.0×25.0 mm). Elution was performed at 60° C. by building up the following gradient at a flow rate of 2 mL/min: 0.1% TFA in acetonitrile/0.1% TFA in water (10/90 v/v) for 10 min, then 0.1% TFA in acetonitrile/0.1% TFA in water/methanol (10/85/5 v/v/v). Melting points were determined with a BUCHI 510 apparatus and are reported uncorrected. ¹H NMR (400 MHz) and ¹³C NMR (100 MHz) spectra were recorded with a BRUKER ADVANCE 400 spectrometer using the residual solvent signal as internal standard. HRMS were obtained from the Mass Spectrometry Service, CRMPO, at the University of Rennes I, France, using a MICROMASS ZABSPEC-TOF spectrometer and a VARIAN MAT311 spectrometer.

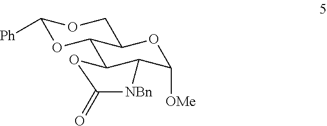 [SMILES: COC1OC2COC(c3ccccc3)OC2C2OC(=O)N(Cc3ccccc3)C12]

To a solution of compound 4 (3.30 g, 10.7 mmol) in dry DMF (15 mL) were added successively K₂CO₃ (7.39 g, 53.4 mmol, 5 eq.) and PHCH₂Br (3.18 mL, 26.7 mmol, 2.5 eq.). The solution was stirred at room temperature for 10 h under Ar atmosphere. A saturated NH₄Cl solution was added and the mixture was extracted with ethyl acetate (3×20 mL). The combined organic layers were dried over MgSO₄, filtered and concentrated. The residue obtained was purified over silica gel column (EtOAc/cyclohexane, 1/3) to afford 5 (3.57 g, 84%) as a white solid; mp 186-188° C.; ¹H NMR (400 MHz, CDCl₃) δ 3.19 (s, 3H, OMe), 3.35 (dd, J=12.0 Hz, 1H, H-2), 3.78-3.85 (m, 2H, H-5, H-6′), 3.98 (q, J=8.0, 12.0 Hz, 1H, H-4), 4.21-4.24 (m, 1H, H-6), 4.45 (s, 2H, NCH2Ph), 4.58 (d, J=4.0 Hz, 1H, H-1), 4.69 (q, J=8.0, 12.0 Hz, 1H, H-3), 5.57 (s, 1H, CHPh), 7.33-7.38 (m, 8H, aromatic), 7.47-7.49 (m, 2H, aromatic); ¹³C NMR (100 MHz, CDCl₃) δ 49.0 (NCH₂Ph), 55.8 (OMe), 62.3 (C-2), 65.6 (C-5), 68.7 (C-6), 73.2 (C-3), 80.4 (C-4), 97.4 (C-1), 101.6 (CHPh), 126.3, 128.5, 128.9, 129.0, 129.2, 129.4, 135.3, 136.7, 158.9 (CO); HRMS (ESI) Calcd. for C₂₂H₂₃NO₆Na [M+Na]⁺: 420.14231. found 420.1411, Calcd. for C₂₂H₂₃NO₆K [M+K]⁺: 436.1162. found 436.1160.

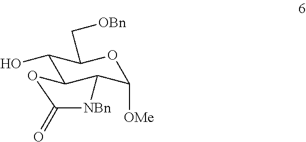 [SMILES: COC1OC(COCc2ccccc2)C(O)C2OC(=O)N(Cc3ccccc3)C12]

To a stirred solution of compound 5 (2.20 g, 5.54 mmol) in dry dichloromethane (10 mL), under Ar atmosphere, was added Et₃SiH (5.47 mL, 66.48 mmol, 12 eq.) and the mixture was cooled to 0° C. To this mixture, BF₃.OEt₂ (1.39 mL, 11.08 mmol, 2 eq.) was added dropwise, then reaction mixture was stirred at 0° C. for 2 h. A saturated NaHCO₃ solution was added and the resulting aqueous mixture was extracted with dichloromethane (3×20 mL). The combined organic layers were dried over MgSO₄, filtered, and concentrated. The residue was purified over silica gel column (EtOAc/cyclohexane, 1/1) to afford 6 (1.79 g, 81%) as a yellow foam; ¹H NMR (400 MHz, CD₃OD) δ 3.13 (s, 3H, OMe), 3.34 (dd, J=4.0, 8.0 Hz, 1H, H-2), 3.57-3.61 (m, 1H, H-5), 3.72-3.73 (m, 2H, H-6, H-6′), 3.89 (t, J=8.0 Hz, 1H, H-4), 4.29 (d, J=12.0 Hz, 1H, NCH₂Ph), 4.37-4.57 (m, 4H, H-3, OCH₂Ph, NCH₂Ph), 4.61 (d, J=4.0 Hz, 1H, H-1), 7.31-7.36 (m, 10H, aromatic); ¹³C NMR (100 MHz, CD₃OD) δ 49.5 (NCH₂Ph), 55.9 (OMe), 62.8 (C-2), 69.7 (C-4), 69.9 (C-6), 74.5 (OCH₂Ph), 75.7 (C-5), 78.4 (C-3), 97.7 (C-1), 128.8, 128.9, 129.1, 129.5, 129.7, 129.8, 137.1, 139.6, 161.4 (CO).

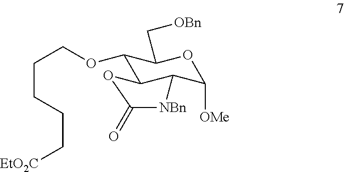 [SMILES: CCOC(=O)CCCCCOC1C(COCc2ccccc2)OC(OC)C2C1OC(=O)N2Cc1ccccc1]

To a solution of compound 6 (2.80 g, 7.01 mmol) in DMF (10 mL) was added NaH (0.56 g, 14.02 mmol, 2 eq., 60% suspension) and the mixture was stirred at room temperature for 10 min under Ar atmosphere. Ethyl 6-bromo hexanoate (2.61 mL, 14.02 mmol, 2 eq.) was added and the mixture was stirred at room temperature for 8 h. A saturated NH₄Cl solution was added and the resulting aqueous mixture was extracted with ethyl acetate (3×20 mL). The combined organic layers were dried over anhydrous MgSO₄, filtered and concentrated. The residue was purified over silica gel column (EtOAc/cyclohexane, 1/3) to afford 7 (3.49 g, 92%) as a yellow foam; ¹H NMR (400 MHz, CDCl₃) δ 1.26 (t, J=8.0 Hz, 3H, Me), 1.27-1.31 (m, 2H, H-3′), 1.45-1.49 (m, 2H, H-2′), 1.59-1.62 (m, 2H, H-4′), 2.27 (t, J=8.0 Hz, 2H, CH₂CO), 3.15 (s, 3H, OMe), 3.27 (dd, J=4.0, 12.0 Hz, 1H, H-2), 3.36-3.37 (m, 1H, OCH₂), 3.58-3.61 (m, 2H, H-5, H-6′), 3.68-3.71 (m, 2H, H-4, H-6), 3.78 (m, 1H, OCH₂), 4.13 (q, J=8.0, 16.0 Hz, 2H, CH₂-ethyl ester), 4.41 (d, 2H, NCH₂Ph), 4.45-4.52 (m, 2H, H-3, OCH₂Ph), 4.55 (d, J=4.0 Hz, 1H, H-1), 4.63 (d, J=12.0 Hz, 1H, OCH₂Ph), 7.29-7.37 (m, 10H, aromatic); ¹³C NMR (100 MHz, CDCl₃) δ 14.4 (Me), 24.9 (C-4′), 25.7 (C-3′), 29.7 (C-2′), 34.4 (CH₂CO), 48.9 (NCH₂Ph), 55.6 (OMe), 60.4 (CH₂-ethyl ester), 61.3 (C-2), 67.9 (C-6), 71.3 (OCH₂), 72.7 (C-5), 73.7 (OCH₂Ph), 75.5 (C-4), 77.4 (C-3), 96.4 (C-1), 127.9, 128.0, 128.4, 128.6, 128.9, 135.4, 138.0, 159.3 (CO-amide), 173.8 (CO-ester); HRMS (ESI) Calcd. for C₃₀H₃₉NO₈Na [M+Na]⁺: 564.2573. found 564.2574, Calcd. for C₃₀H₃₉NO₈K [M+K]⁺: 580.2313. found 580.2345, Calcd. for C₂₉H₃₉NO₆Na [M-CO₂+Na]⁺: 520.2675. found 520.2688.

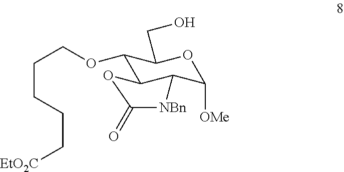 [SMILES: CCOC(=O)CCCCCOC1C(CO)OC(OC)C2C1OC(=O)N2Cc1ccccc1]

A mixture of compound 7 (3.20 g, 5.91 mmol) and Pd/C (10%) (1.18 mmol, 0.2 eq.) in EtOH (10 mL) was stirred under hydrogen atmosphere for 12 h, filtered through a pad of celite and concentrated. The residue was purified by chromatography over silica gel column (EtOAc/pentane, 2/1) to afford 8 (2.61 g, 98%) as a yellow foam; ¹H NMR (400 MHz, CDCl₃) δ 1.25 (t, J=8.0 Hz, 3H, Me), 1.36-1.38 (m, 2H, H-4′), 1.55-1.65 (m, 4H, H-2′, H-3′), 2.29 (t, J=8.0 Hz, 2H, CH₂CO), 3.15 (s, 3H, OMe), 3.21 (dd, J=12 Hz, 1H, H-2), 3.49-3.53 (m, 214, H-5, OCH₂), 3.67 (t, J=8.0 Hz, 1H, H-4), 3.76 (d, 21-1, H-6, H-6′), 3.83-3.87 (m, 1H, OCH₂), 4.12 (q, J=8.0, 16.0 Hz, 2H, CH₂-ethyl ester), 4.41 (d, 2H, NCH₂Ph), 4.50-4.55 (m, 2H, H-1, H-3), 7.30-7.36 (m, 5H, aromatic); ¹³C NMR (100 MHz, CDCl₃) δ 14.2 (Me), 24.6 (C-4′), 25.5 (C-3′), 29.4 (C-2′), 34.2 (CH₂CO), 48.6 (NCH₂Ph), 55.4 (OMe), 60.2 (CH₂-ethyl ester), 61.0 (C-6), 61.2 (C-2), 71.0 (OCH₂), 73.3 (C-5), 75.4 (C-4), 77.1 (C-3), 96.2 (C-1), 128.2, 128.6, 128.7, 135.2, 159.1 (CO, amide), 173.8 (CO, ester).

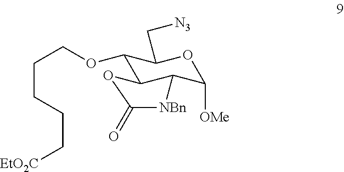 [SMILES: CCOC(=O)CCCCCOC1C(CN=[N+]=[N-])OC(OC)C2C1OC(=O)N2Cc1ccccc1]

To a solution of compound 8 (1.60 g, 3.54 mmol) in dry pyridine (10 mL) was added TsCl (1.69 g, 8.85 mmol, 2.5 eq.) and the mixture was stirred at ambient temperature under Ar atmosphere. After completion of the reaction (TLC), saturated NaHCO₃ solution was added and the resulting aqueous mixture was extracted with ethyl acetate (3×10 mL). The combined organic layers were dried over anhydrous MgSO₄, filtered and concentrated. The residue was purified over silica gel column (EtOAc/pentane, 1/2) to afford the desired compound (2.06 g, 96%) as a yellow oil.

To a solution of this tosylated compound (1.90 g, 3.14 mmol) in DMF (10 mL) was added NaN₃ (2.04 g, 31.4 mmol, 10 eq.), then the solution was heated at 80° C. for 3 h under Ar atmosphere. A saturated NH₄Cl solution was added and the resulting aqueous mixture was extracted with ethyl acetate (3×10 mL). The combined organic layers were dried over anhydrous MgSO₄, filtered and concentrated under reduced pressure. The residue was purified over silica gel column (EtOAc/pentane, 1/3) to afford 9 (1.36 g, 91%) as a yellow gummy liquid; ¹H NMR (400 MHz, CDCl₃) δ 1.24 (t, J=8.0 Hz, 3H, Me), 1.32-1.36 (m, 2H, H-3′), 1.53-1.56 (m, 2H, H-2′), 1.58-1.64 (m, 2H, H-4′), 2.28 (t, J=8.0 Hz, 2H, CH₂CO), 3.13 (s, 3H, OMe), 3.24 (dd, J=4.0, 12.0 Hz, 1H, H-2), 3.43-3.47 (m, 3H, H-6, H-6′, OCH₂), 3.55 (t, J=8.0, 12.0 Hz, 1H, H-4), 3.62-3.65 (m, 1H, H-5), 3.81-3.87 (m, 1H, OCH₂), 4.11 (q, J=8.0, 16.0 Hz, 2H, CH₂-ethyl ester), 4.34 (d, J=16.0 Hz, H-1, NCH₂Ph), 4.45-4.51 (m, 3H, H-1, H-3, NCH₂), 7.30-7.36 (m, 5H, aromatic); ¹³C NMR (100 MHz, CDCl₃) δ 14.4 (Me), 24.8 (H-4′), 25.7 (H-3′), 29.6 (H-2′), 34.3 (CH₂CO), 48.9 (NCH2Ph), 51.0 (C-6), 55.7 (OMe), 60.4 (CH₂-ethyl ester), 61.5 (C-2), 71.2 (OCH₂), 72.5 (C-5), 76.5 (C-4), 76.9 (C-3), 96.3 (C-1), 128.4, 128.8, 128.9, 135.3, 159.0 (CO-amide), 173.7 (CO-ester).

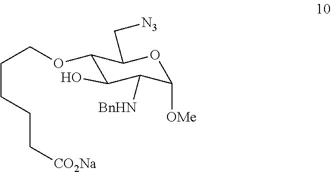

A solution of compound 9 (1.10 g, 2.31 mmol) in a mixture of 1M NaOH/1,4-dioxan (5/5 mL, v/v) was heated at 80° C. for 20 h. The mixture was diluted with ethyl acetate and washed with brine. The separated aqueous layer was repeatedly washed with ethyl acetate. The combined organic layers were dried over anhydrous MgSO₄, filtered and concentrated. The residue obtained was purified over silica gel column (CH₂Cl₂/MeOH, 9/1) to afford 10 (0.72 g, 74%) as a yellow gummy liquid; ¹H NMR (400 MHz, CDCl₃) δ 1.37-1.43 (m, 2H, H-3′), 1.53-1.64 (m, 4H, H-2′, H-4′), 2.30 (t, J=4.0, 8.0 Hz, 2H, CH₂CO), 2.70 (dd, J=4.0, 12.0 Hz, 1H, H-2), 3.13 (t, J=8.0 Hz, 1H, H-4), 3.31 (s, 3H, OMe), 3.40 (dd, J=8.0, 12.0 Hz, 1H, H-6′), 3.48 (dd, J=12.0 Hz, 1H, H-6), 3.51-3.55 (m, 1H, OCH₂), 3.62-3.66 (m, 1H, H-5), 3.76 (t, J=8.0, 12.0 Hz, 1H, H-3), 3.88-3.94 (m, 1H, OCH₂), 3.96 (s, 2H, NCH₂Ph), 4.59 (d, J=4.0 Hz, 1H, H-1), 6.63 (bs, NH), 7.32-7.37 (m, 5H, aromatic); ¹³C NMR (100 MHz, CDCl₃) δ 24.9 (C-4′), 25.6 (C-3′), 30.0 (C-2′), 35.2 (CH₂CO), 51.6 (NCH₂Ph), 51.7 (C-6), 55.5 (OMe), 61.6 (C-2), 70.2 (C-5), 72.2 (C-3), 72.3 (OCH₂), 78.9 (C-4), 97.1 (C-1), 128.2, 128.8, 129.0, 137.6, 179.5 (CO).

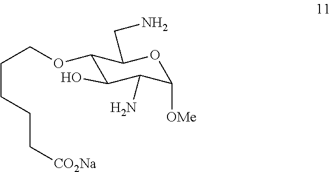

To a solution of compound 10 (0.64 g, 1.44 mmol) in a of MeOH/H₂O (9/1 mL) mixture were added Pd(OH)₂/C (20%) (0.04 g, 0.29 mmol, 0.2 eq.) and HCO₂NH₄ (0.45 g, 7.20 mmol, 5 eq.). The mixture was refluxed for 1.5 h, filtered through a pad of celite and concentrated to afford 11 (0.45 g, 95%) as a white crystalline solid; mp 87-89° C.; ¹H NMR (400 MHz, D₂O) δ 1.30-1.35 (m, 2H, H-4′), 1.51-1.60 (m, 4H, H-2′, H-3′), 2.16 (t, J=8.0 Hz, 2H, CH₂CO), 2.93 (dd, J=4.0, 12.0 Hz, H-1, H-2), 3.11-3.22 (m, 2H, H-4, H-6′), 3.37 (dd, J=4.0, 12.0 Hz, 1H, H-6), 3.40 (s, 3H, OMe), 3.61-3.70 (m, 2H, H-3, OCH₂), 3.81-3.87 (m, 21-1,1′-5, OCH₂), 4.85 (s, 1H, H-1); ¹³C NMR (100 MHz, D₂O) δ 25.0 (C-4′), 25.4 (C-3′), 28.9 (C-2′), 37.3 (CH₂CO), 40.3 (C-6), 54.4 (C-2), 55.4 (OMe), 67.1 (C-5), 72.0 (C-3), 73.1 (OCH₂), 79.7 (C-4), 98.5 (C-1), 183.6 (CO).

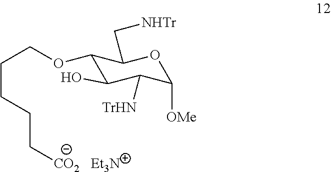

A solution of compound II (0.22 g, 0.67 mmol) in a DMF/triethylamine (7/1 mL) mixture, under Ar atmosphere, was stirred at room temperature for 30 min, and then a solution of trityl chloride (0.93 g, 3.35 mmol, 5 eq.) in a DMF/triethylamine (5/1) mixture was added. The resulting solution was stirred at room temperature for 8 h. A saturated NH₄Cl solution was added and the resulting aqueous mixture was extracted with ethyl acetate (3×10 mL). The combined organic layers were dried over anhydrous MgSO₄, filtered and concentrated. The residue obtained was purified over silica gel column (EtOAc/cyclohexane, 1/8 containing triethylamine) to give the tri-tritylated compound (0.47 g, 68%) as a white amorphous solid; HRMS (ESI) Calcd. for C₇₀H₆₉N₂O₆ [M+H]⁺: 1033.5156. found 1033.5163.

A solution of this compound (0.45 g, 0.43 mmol) in a mixture 1M NaOH/dioxane (4/4 mL, v/v) was heated at 80° C. for 6 h, then diluted with ethyl acetate and washed with a saturated solution of NaCl. The separated aqueous layer was repeatedly washed with ethyl acetate. The combined organic layers were dried over anhydrous MgSO₄, filtered and concentrated. The obtained residue was purified over silica gel column (CH₂Cl₂/MeOH, 9/1) to give 12 (0.33 g, 85%) as white crystalline solid; mp 107-109° C.; ¹H NMR (400 MHz, CDCl₃) δ 1.21-1.27 (m, 2H, H-4′), 1.30 (t, J=4.0, 8.0 Hz, 9H, Et₃N), 1.41-1.44 (m, 2H, H-3′), 1.55-1.57 (m, 2H, H-2′), 1.97-1.98 (m, 1H, H-6′), 2.19 (t, J=8.0 Hz, 2H, CH₂CO), 2.50-2.53 (m, 1H, H-6), 2.77 (d, J=4.0 Hz, 1H, H-1), 2.81 (dd, J=4.0, 8.0 Hz, 1H, H-2), 2.92 (q, J=4.0, 8.0 Hz, 1H, H-4), 2.99 (s, 3H, OMe), 3.05 (q, J=8.0, 12.0 Hz, 6H, Et₃N), 3.28-3.31 (m, 1H, OCH₂), 3.64 (m, 1H, H-5), 3.76-3.83 (m, 2H, H-3, OCH₂), 7.16-7.30 (m, 20H, aromatic), 7.40-7.42 (m, 5H, aromatic), 7.56-7.58 (m, 5H, aromatic); ¹³C NMR (100 MHz, CDCl₃) δ 8.9 (Me, Et₃N), 25.6 (C-4′), 26.1 (C-3′), 30.3 (C-2′), 36.1 (CH₂CO), 45.1 (C-6), 45.4 (CH₂, Et₃N), 54.2 (OMe), 58.1 (C-2), 66.4 (CO trityl), 70.2 (C-5), 70.3 (CO trityl), 71.9 (OCH₂), 73.8 (C-3), 80.7 (C-4), 97.5 (C-1), 126.3, 126.7, 126.8, 127.8, 127.9, 128.0, 128.1, 128.3, 128.8, 129.0, 129.1, 146.2, 147.0, 148.7, 179.1 (CO).

Procedure for the synthesis of glucosamine-PNA conjugates 2a and 2b is as follows.

To a solution of the protected glucosamine derivative 12 (39 mg, 20 μmol) in dry DMF (5000 μL), under Ar, were added 1-[3-(dimethylaminopropyl)]-3-ethylcarbodiimide hydrochloride (5.7 mg, 30 μmol) and 1-hydroxybenzotriazole (HOBt) (4.0 mg, 30 μmol). The resulting solution was stirred for 15 min. and then the protected PNA on its solid support was added under Ar (PNA synthesis at 2 μM scale). The mixture was stirred at room temperature for 1 h and filtered.

The neamine PNA conjugate was then cleaved from the solid support with concomitant deprotection by treatment with TFA/anisole (1/1) for 1 h. The mixture was filtered and, then, diethyl ether was added to the solution to precipitate the PNA.

HPLC purification was carried out on a C₁₈ reversed-phase column (Macherey-Nagel, 10.0×25.0 mm). Elution was performed at 60° C. by building up the following gradient at a flow rate of 2 mL/min: 0.1% TFA in acetonitrile/0.1% TFA in water (10/90 v/v) for 10 min, then 0.1% TFA in acetonitrile/0.1% TFA in water/methanol (10/85/5 v/v/v).

MALDI-TOF MS, conjugate 2a, m/z found, 4575, calcd for C₁₈₃H₂₃₉N₉₁O₅₄, 4574.88, (non coupled and free PNA: 4286.71).

### Biochemistry and Biological Assays

### Preparation of HIV-1 TAR RNA

The labeled and unlabeled run-off transcript of HIV-1 TAR RNA was prepared from plasmid pEM-7 linearized with HindIII using standard methods. The labeled transcript was purified by 10% polyacrylamide-urea gel electrophoresis. The radioactive band was excised from the gel, extracted in 0.5 M ammonium acetate, desalted on a NAP-10 column (Pharmacia, Inc., Piscataway, N.J.), lyophilized, and dissolved in 10 mM Tris-HCl, pH 7.8, 60 mM KCl and 10 mM DTT and stored at −70° C. The specific radioactivity of the resulting purified transcript was determined by A₂₆₀ absorbance and Cerenkov counting.

### The Binding Affinity of PNA_{TAR}-Glucosamine to TAR RNA

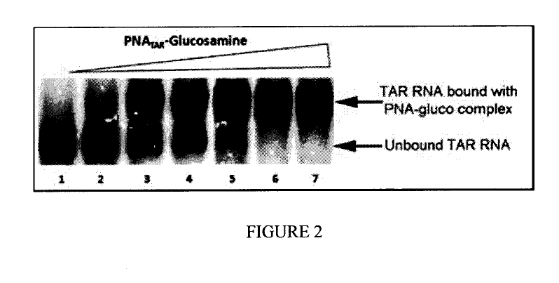

The binding affinity of PNA_{TAR}-glucosamine conjugate for TAR RNA was evaluated by gel mobility shift analysis according to standard methods. In brief, anti-TAR PNAs at varying molar ratios were incubated with 10 nM of ³²P-labeled TAR RNA transcript (5000 Cerenkov cpm) for 30 min at 37° C. in a binding buffer containing 50 mM Tris-HCl, pH 7.8, 60 mM KCl, 5.0 mM MgCl₂, 10 mM DTT, 10% glycerol, 0.01% NP-40 and 500 ng of r (1-C), in a final volume of 15 μl. Three microliters of RNA gel loading dye (0.27% bromophenol blue and 30% glycerol) was added to the samples prior to loading. Samples were then subjected to polyacrylamide DNA retardation analysis on a native 6% polyacrylamide gel in Tris-Borate buffer. The gels were routinely pre-run at 120 V for 30 minutes at 4° C. in Tris-Borate buffer, pH 8.2. The RNA-PNA complexes were resolved at a constant voltage of 120 V at 4° C. for 3 hours and detected by phosphorimaging (FIG. 2). Lane 1 through 7 represents molar ratios of PNA_{TAR}-Glucosamine to TAR RNA of 0.0, 0.2, 0.5, 0.8, 1.0, 2.0 and 5.0 respectively. Lane 1 TAR RNA was considered a control. As seen in the Figure, the binding of PNA-glucosamine to its target sequence is stoichiometric. At the 1:0.5 ratio of target RNA to PNA-glu, 50% shift in the RNA mobility is seen (lane 3) while 100% shift in the mobility is seen at 1:1 ratio and above (lanes 5-7).

FIG. 2 shows the binding affinity of PNA_{TAR}-glucosamine conjugate to its target sequence. The gel mobility shift assay was performed to assess the binding affinity of the PNA_{TAR}-glucosamine conjugate. The PNA_{TAR}-glucosamine conjugate was incubated at varying concentration with 10 nM of internally ³²P-labeled TAR RNA transcript in binding buffer for 30 min at room temperature. The incubated samples were loaded on a 8% native polyacrylamide gel and RNA:PNA complex was separated from free RNA by running at 150 V for 3 h. Lane 1 represents TAR RNA alone; lanes 2 to 7 represent increasing ratios of TAR RNA to PNA_{TAR}-glucosamine conjugate from 0.2, 0.5, 0.8, 1.0, 2.0 and 5.0 respectively.

Cellular Uptake of Fluorescein labeled PNA_{TAR} Glucosamine conjugate in CEM Cells

The CEM cells were cultured in RPMI1640 contains 10% fetal bovine serum, 5% penicillin and streptomycin at 37° C. in a 5% CO₂ environment. Cells were harvested, washed with PBS and resuspended in RPMI1640 contains only 1% FBS at cell density of 0.5×10⁶ cells/ml. Cells were aliquoted in 12-well plate at 0.5×10⁶ cells/ml and incubated for 30 min at 37° C. Fluorescein tagged PNA_{TAR} Glucosamine conjugate were added to cells at varying concentration (50 nM-500 nm) and final volume were adjusted to 1 ml by adding same media. At different time points cells were harvested by centrifuge (rocking angle) and washed with PBS twice and resuspended in 1 ml PBS for FACScan on Becton Dickinson flow cytometer. 0.5% propidium Iodide were added to all the sets and quickly scanned to make certain that uptake occurred only in live cells. Cell Quest Pro software (Becton Dickinson) was used to acquire and analyze events detected by FL1 detector (for FITC), which excluded FL3 detector (Propidium iodide). Graphs were generated using GraphPad Prism.

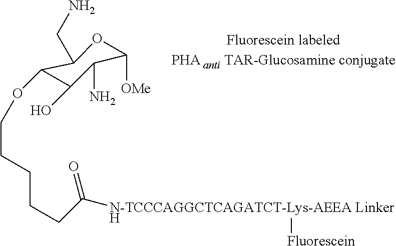

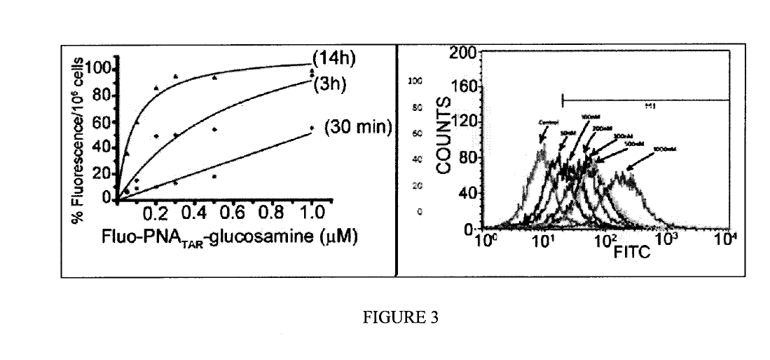

FIG. 3 depicts flow cytometry analysis of uptake of anti-HIV-1 PNA_{TAR}-glucosamine conjugate. CEM cells (0.5×10⁶) were incubated with increasing concentrations of PNA_{TAR}-glucosamine conjugate for 1 min at 4° C. and 37° C. After washing thoroughly with 1×PBS, the cells were resuspended in RPMI media with 2% FBS. The right panel shows flow cytometry data carried out in presence of propidium iodide, while the left panel shows percent FITC-uptake per 10⁴ cells as a function of concentration of PNA_{PBS}-MTD peptide conjugates.

### Inhibition of Reverse Transcription of Target RNA

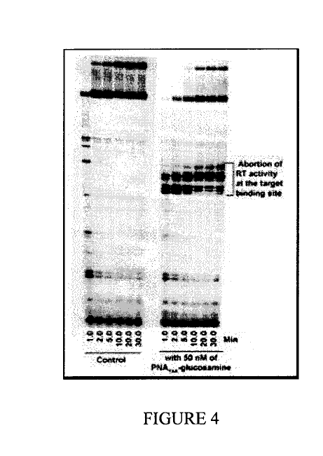

Since PNA-RNA or PNA-DNA duplexes exhibit higher melting temperatures than corresponding RNA-DNA or DNA-DNA duplexes, it was determined whether the PNA_{TAR}-glucosamine conjugate was able to block reverse transcription of the HIV-1 TAR. Blocking reverse transcription would have multiple effects on viral replication besides influencing Tat-mediated transactivation. For this reason, 10 nM of TAR RNA primed with 10 nM of labeled 17-mer DNA primer was incubated in the absence or presence of the conjugate at 37° C. followed by initiation of reverse transcription by HIV-1 reverse transcriptase at different time points. The results of these experiments indicated that prominent pauses in reverse transcription occurred at the site targeted by the PNA-glucosamine conjugate (FIG. 4). These results show that the anti-TAR PNA-glucosamine conjugate binds to its target site on TAR RNA and blocks its reverse transcription, probably by inhibiting the strand displacement activity of HIV-1 reverse transcriptase.

As noted above, FIG. 4 provides the results of the primer extension assay with TAR-RNA template in the presence PNA_{TAR}-glucosamine conjugate. Ten nM of annealed template primer was incubated with 5 nM concentrations of NA_{TAR}-glucosamine at 37° C. Reverse transcription reactions were initiated by addition of 100 μM of dNTPs and 100 nM of HIV-1 RT. Reactions were carried out at 37° C. and stopped after indicated time points by addition of 2× Sanger's gel loading dye. Reaction products were resolved on an 8% denaturing polyacrylamide-urea gel. Control samples represent reverse transcription in the absence of PNA_{TAR}-peptide. The accumulation of aborted RT product at the target binding site is indicated.

### Isolation of HIV-1 Virions and Infection of CEM T-Lymphocytes

The pseudotyped HIV-1 virions were isolated from the culture supernatant of 293T cells transfected with pHIV-1_{JR-CSF-Luc}env (−) and pVSV-G clones. The culture supernatant (500 mL) was filtered through 0.45 μm pore size membrane and centrifuged at 70,000 g for 45 minutes. The pelleted virions were resuspended in fresh culture medium containing 10% fetal calf serum and stored at −80° C. HIV-1 virions were quantified by determining the RNA copy number in the sample using NUCLISENS HIV-1-QT Amplication Kit (Organon Teknika, Durham, N.C.). The virion number was also extrapolated from the p24 concentration considering that 2000 copies of p24 are present per virion particle. The virion number estimated from the RNA copy number was in agreement with the number determined by p24 quantification (1 pg p24 per 12500 virions).

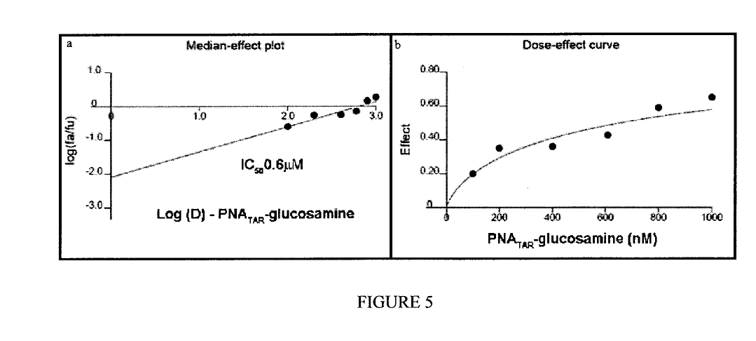

FIG. 5 is a graphical depiction of the dose effect curve of virucidal activity of anti-HIV-1 PNA_{TAR}-glucosamine conjugate: The pseudotyped HIV-1 virions (equivalent to 100 ng of p24) were first incubated with increasing concentrations of PNA_{TAR}-glucosamine for 2 h. The pretreated virions were then used to infect the CEM cells as described herein. The infected cells were grown for 48 h and the extent of infection was determined by determining the levels of luciferase reporter enzyme in cell lysate. Dose effect curves and Median-effect plots were calculated using Calcusyn software (Biosoft) as described in Materials and Methods. FIG. 5(a) shows dose effect curves for virucidal activity of PNA_{PBS}-MTD peptide conjugates; FIG. 5(b) shows dose effect curves for virucidal activity of PNA_{A-Loop}-MTD peptide conjugates.

### Anti HIV-1 Activity of PNA_{TAR}-Glucosamine Conjugate in Cell Culture

Lymphocyte CEM (12D7) cells were maintained in complete RPMI-1640 medium supplemented with 10% fetal bovine serum (FBS), 100 U/mL of penicillin and 100 μg/mL of streptomycin at 37° C. in 5% CO₂ containing humidified air. CEM cells (0.5×10⁶) were infected with pseudotyped HIV-1 S1 strain and incubated in the presence of indicated concentration of PNA_{TAR}-glucosamine conjugate or naked PNA. Cells were harvested 48 hours after infection, lysed and assayed for luciferase activity according to standard methods.

The foregoing examples and description of the preferred embodiments should be interpreted as illustrating, rather than as limiting the present invention as defined in the specification. All variations and combinations of the features above are intended to be within the scope of the specification.

### PNA-Glucosamine Conjugate is Efficiently Taken Up by Cells.

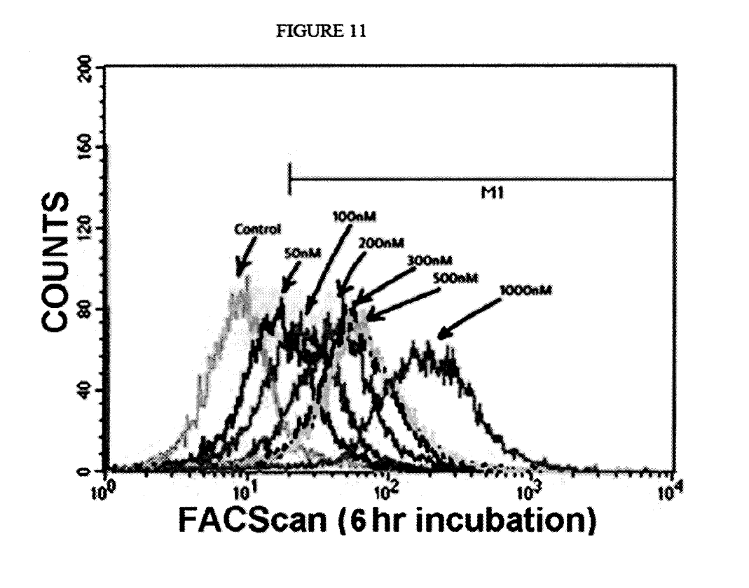

For uptake studies, we prepared conjugate of 15 mer PNA-glucosamine that contained a fluorescein probe attached to the PNA molecule. The fluorescein-labeled conjugate was dissolved in water and its concentration determined by absorption of fluorescein at 490 nm (ε=67,000); the concentration of PNA was determined by absorption at 260 nm (ε=171,200). Similar molar concentrations obtained by these two methods not only established their accuracy but also suggested the absence of free fluorescein in the preparation. Using this fluoresceintagged PNA-glucosamine conjugate, we did a series of experiments to determine the uptake efficiency of the conjugate using flow cytometry (FIG. 11). Fluorescein-tagged naked (unconjugated) PNA was used as a control (FIG. 11). The uptake of PNA-glucosamine conjugate in Huh7.5 cells occurred efficiently (FIG. 11). At a concentration of conjugate as low as 200 nM, nearly 80% of the cells were fluorescence positive within 6 h of incubation. Uptake of Flu-tagged naked unconjugated PNA at 1,000 nM concentration was negligible (FIG. 11, control).

### In-Situ Capture of HCV Genomic RNA-Protein Complex by PNA 4′-Glucosamine Conjugate Targeted to HCV Core Coding Region Downstream to the 5′NTR.

We used both MH14 cells (HCV positive) carrying stably replicating HCV subgenomic replicons and cured MH 14 cells (HCV negative) devoid of HVC replicons in our in-situ affinity capture experiment by anti-HCV PNA-glucosamine conjugate. The anti-HCV PNA-glucosamine conjugate was complementary to nucleotide sequence 342-356 downstream to the 5′NTR and was biotinylated through lysine residue at the C-terminus of PNA. We gently washed the subconfluent cells with cold buffer containing 150 mM sucrose and 30 mM HEPES at pH 7.4, 33 mM NH₄Cl, 7 mM KCl, and 4.5 mM magnesium acetate. We layered the washed cells with reticulocyte buffer containing 1.6 mM Tris-acetate, pH 7.8, 80 mM KCl, 2 mM Mg acetate, 0.25 mM ATP, 0.1 mM dithiothreitol, 10 U of RNasin) containing 0.5 μM of biotinylated anti-HCV PNA-glucosamine. We also used a biotinylated scrambled PNAglucosamine conjugate as a negative control. After incubation at the room temperature for 2 h, we washed the cells with the same buffer and then scraped them from each plate and lysed them on ice. We centrifuged the lysed cells for 10 min at low speed (7,000×g). The supernatant (S7 fraction) was incubated with 150 μl of paramagnetic streptavidin beads on ice for 1 h to capture the HCV RNA-protein complex bound to biotinylated PNA-glucosamine conjugate. We washed the beads 6 times with the reticulocyte buffer containing 500 mM NaCl. The captured (+) strand HCV RNA-protein complex was then eluted from the beads by adding 30 μl of binding buffer and 30 μl of 2×SDS gel loading dye to the washed beads and heating at 950 C for 5 minutes before magnetic separation of beads from eluted proteins. Samples were loaded on an 8%-16% gradient SDS page gel and the gel was stained with Sypro Ruby dye (Molecular Probes) for visualization of protein bands.

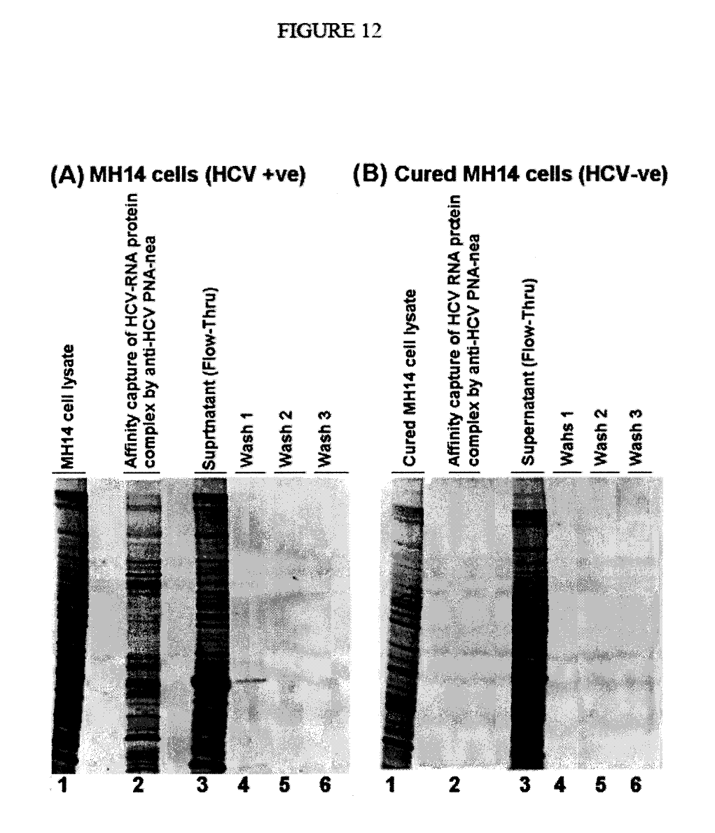

As shown in FIG. 12A, the anti-HCV PNA-glucosamine conjugate could efficiently penetrate the cells and capture the HCV-RNA protein complex in-situ from MH 14 cells (FIG. 12A; lane 2). A number of proteins bands associated with the captured HCV (+) RNA genome from MH14 cells could be seen in the gel (FIG. 12A, lane 2). The binding of the RNA-protein complex to the PNA probe was tight enough to withstand washing with 0.5M salt as no protein bands could be seen in the washes (FIG. 12A, lanes 4-6). We excised each protein band from the gel (FIG. 12A, lane 2) and processed for LC MS/MS analysis for their identification using proteomics technology. In contrast, affinity capture from cured-MH14 culls devoid of replicating HCV RNA, exhibited complete absence of proteins in the gel (FIG. 12B, lane 2). These results suggest that PNA-glucosamine conjugates targeted to HCV genome are specific to their target sequence on the viral genome and neither they recognize off-site target on cellular RNA nor do they have any affinity for cellular proteins.

### Functional Validation of Anti-HCV PNA-Glucosamine Conjugate Targeted to the Translation 20 Initiation Window in Domain IV of HCV 5′NTR.

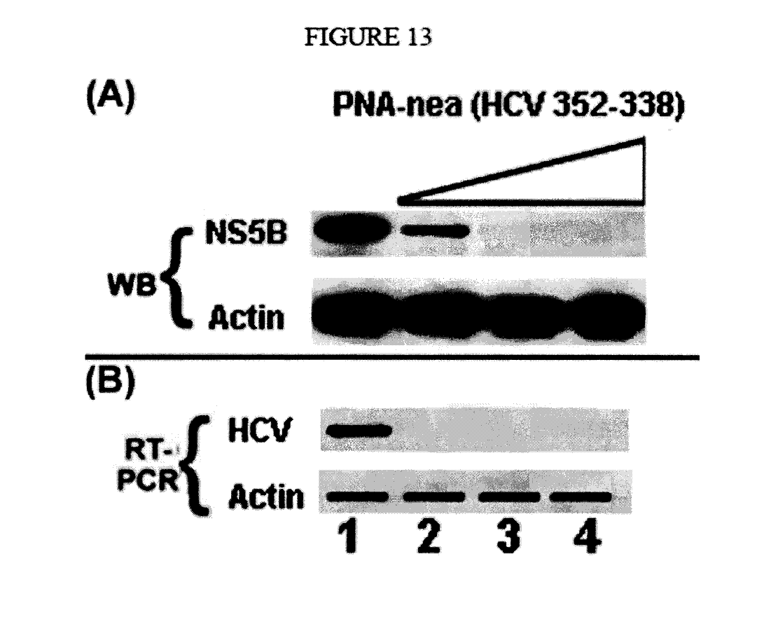

Since PNA-glucosamine efficiently penetrates liver cells, we did a functional assay to determine whether PNA-glucosamine conjugate targeted to the translation initiation window in domain IV of HCV 5′ NTR is able to block viral replication and translation when added in cell culture medium. MH14 cells carrying replicating HCV subgenomic replicons were grown in the presence of different concentrations of PNA-glucosamine conjugate targeted to nucleotide sequence 352-338 in domain IV of the HCV 5′ NTR. Cells were harvested after 72 h. The presence of viral protein (NS5B) was detected by Western blotting of cell lysate; the viral RNA was analyzed by RT PCR of total RNA isolated from the cells. The results were exciting: Both HCV translation (FIG. 13A) and replication (FIG. 13B) of HCV were efficiently blocked at a subnanomolar concentration of the conjugate (lanes 2-4). These results clearly indicate that anti-HCV-PNA-glucosamine conjugate is a candidate for use in a drug to intervene in HCV replication and translation. Neither unconjugated PNA nor scrambled PNA-glucosamine conjugate affected viral replication and translation.

### Endogenous HCV Replication Activity in MH14 Cells is Strongly Inhibited by PNAglucosamine Conjugated Complementary to Nucleotide Sequence 1-16 in the (−) Strand RNA.

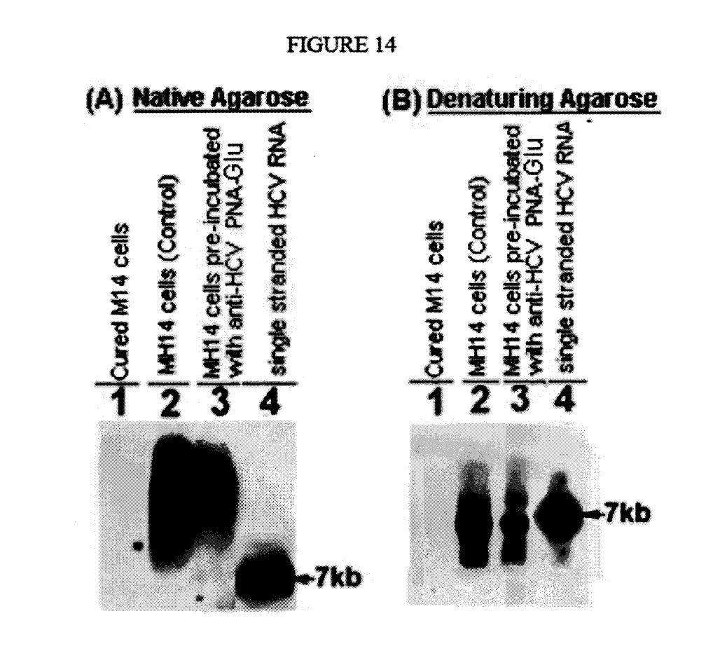

We examined in vitro endogenous HCV replication activity in MH14 cells pre-incubated with PNA-glucosamine-RNA1-16 conjugate. The MH14 cells were pre-incubated with or without PNA-glucosamine-RNA1-16 conjugate for 8 hours. The cells were harvested, lysed and examined for endogenous replication activity in the cell-free lysate as described by Ali and Siddiqui, (2002). As shown in FIG. 14, the endogenous HCV replication activity in MH14 cells was drastically reduced when MH14 cells were pre-incubated with PNA-glucosamine-RNA1-16 (FIG. 14; lane 3) as compared to the control MH14 cells (lane 2). Cell lysate from cured MH14 cells were devoid of such activity (lane 1). The products size on the native agarose gel were much longer than the replicon size while on denaturing agarose gel they migrated as 7 kb HCV replicon (FIG. 14B). This could be due to association of newly synthesized RNA with the endogenous RNA template.

### REFERENCES

The following are herein incorporated by reference in their entirety
1. J.-L. Décout, V. N. Pandey, E. Riguet. PNA-neamine conjugates and methods for producing and using the same. US patent 15 Dec. 2003, US 20070225239, WO 2005060573. Université Joseph Fourier/University of Medicine of New Jersey, Newark, USA/CNRS.
2. J.-L. Décout, I. Das, J. Désiré, I. Baussanne, M.-P. Mingeot-Leclercq; Dérivés de 6-amino-6-désoxyglucosamine et leur utilisation comme agents antibactériens; Demande de brevet francais déposée le 9 novembre 2010.
3. E. Riguet, S. Tripathi, B. Chaubey, J. Désiré, V. N. Pandey, J.-L. Decout. A peptide nucleic acid-neamine conjugate that targets and cleaves HIV-1 TAR RNA inhibits viral replication. J. Med. Chem. 2004, 47, 4806-4809.
4. B. Chaubey, S. Tripathi, J. Désiré, I. Baussanne, J.-L. Decout, V. N. Pandey. Mechanism of RNA cleavage catalyzed by sequence specific polyamide nucleic acid-neamine conjugate. Oligonucleotides 2007, 17, 302-313.
5. Nielsen, et al. (1991) Science 254:1497-1500; Hyrup and Nielsen (1996) Bioorg. Med. Chem. 4:5-23; Uhlmann, et al. (1998) Angew. Chem. Int. Ed. 37:2796-2823.
6. Koppeihus and Nielsen (2003) Adv. Drug Deliv. Rev. 55:267-280.
7. Kaushik, et al. (2002) J. Virol. 76:3881-3891.
8. Chaubey et al., (2005) VIROLOGY 331: 418-428.
9. Tripathi et al., (2005) NUCLEIC ACIDS RES. 33, 4345-4356.
10. Tripathi et al., VIROLOGY 363, 91-103 (2007).
11. Moazed and Noller (1987) Nature 327:389-394.
12. Das et al., J. Med. Chem., 2012, 55 (13), pp 6021-6032

1. A compound comprising a nucleic acid oligomer and a glucosamine conjugated thereto.

2. The compound of claim 1, wherein said glucosamine is 6-aminoglucosamine.

3. The compound of claim 1, wherein said nucleic acid oligomer is a peptide nucleic acid (PNA) oligo.

4. The compound of claim 2, wherein said nucleic acid oligomer is a peptide nucleic acid (PNA) oligo.

5. The compound of claim 1, wherein said nucleic acid oligomer hybridizes selectively to viral RNA.

6. The compound of claim 5, wherein said viral RNA is HIV-1 RNA.

7. The compound of claim 5, wherein said nucleic acid oligomer hybridizes HIV TAR RNA.

8. The compound of claim 7, wherein said nucleic acid oligomer comprises the sequence TCCCAGGCTCAGATCT (SEQ ID NO: 1).

9. The compound of claim 5, wherein said viral RNA is HCV RNA.

10. The compound of claim 9, wherein said nucleic acid oligomer hybridizes Domain IV of HCV 5′NTR.

11. The compound of claim 10, wherein said nucleic acid oligomer comprises the sequence TTCGTGCTCATGGTG (SEQ ID NO: 2).

12. The compound of claim 1, selected from the group consisting of

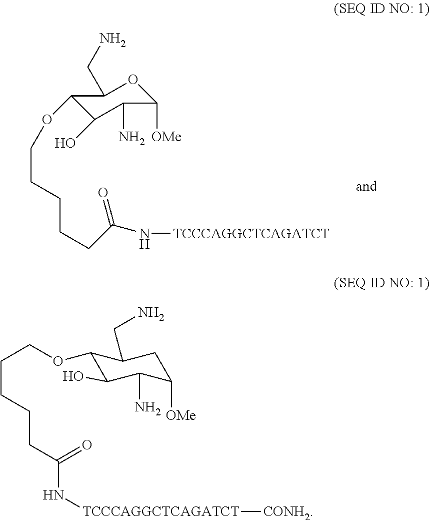

13. The compound of claim 1 which is:

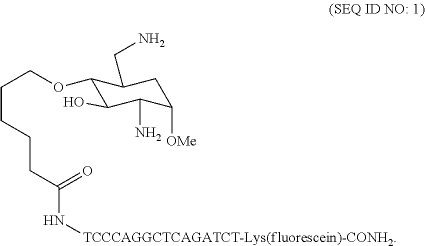

14. A composition comprising a compound of formula I and a pharmaceutically acceptable carrier or excipient.

15. A method of treating a disease or disorder in a mammal mediated by a protein or nucleic acid comprising administering to said mammal an effective amount of a conjugate compound of the invention wherein the nucleic acid of the conjugate compound hybridizes to the nucleic acid mediating said disease or disorder or to the nucleic acid encoding the protein mediating said disease or disorder.

16. The method of claim 15, wherein said disease or disorder is a viral infection.

17. The method of claim 16, wherein said viral infection is an HIV or HCV infection.

18. The method of claim 15, wherein said disease or disorder is cancer.

19. The method claim 18, wherein the nucleic acid oligomer hybridizes mRNA encoding telomerase.

20. The method of claim 15, wherein nucleic acid oligomer is a PNA oligomer and the glucosamine is 6-aminoglucosamine.


SEQUENCE LISTING (5 sequences)

SEQ ID NO: 1 - DNA, 16 bp, Artificial Sequence
  Description of Artificial Sequence Synthetic oligonucleotide
tcccaggctcagatct

SEQ ID NO: 2 - DNA, 15 bp, Hepatitis C virus
ttcgtgctcatggtg

SEQ ID NO: 3 - RNA, 377 nt, Hepatitis C virus
gccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaa

SEQ ID NO: 4 - RNA, 247 nt, Artificial Sequence
  Description of Artificial Sequence Synthetic polynucleotide
acuccccaaccgaugaacggggagcuaaacacuccaggccaauaggccauccuguuuuuuucyyyyyyyyyyyyyyyyyyyyyyyyyyyyyyyyyyyyyyyyyyyyyyyyyyyyyyyyyyyyyyyyyyyyyyyyyyyyyyyyyyycuuugguggcuccaucuuagcccuagucacggcuagcugugaaagguccgugagccgcuugacugcagagagugcugauacuggccucucugcagaucaagu

SEQ ID NO: 5 - DNA, 15 bp, Artificial Sequence
  Description of Artificial Sequence Synthetic oligonucleotide
cagtatcagcactct